(19) United States
(12) Patent Application Publication
(10) Pub. No.: US 2022/0214283 A1
(43) Pub. Date: Jul. 7, 2022

(54) METHOD FOR IDENTIFYING GENERATION SOURCES OF FINE PARTICULATES IN ATMOSPHERE

(71) Applicants: Kazuichi HAYAKAWA, Ishikawa (JP); SHIMADZU CORPORATION, Kyoto (JP)

(72) Inventors: Kazuichi HAYAKAWA, Ishikawa (JP); Keiko MATSUMOTO, Kyoto (JP); Tadayuki YAMAGUCHI, Kyoto (JP)

(21) Appl. No.: 17/604,461

(22) PCT Filed: May 16, 2019
(86) PCT No.: PCT/JP2019/019436

§ 371 Date: Oct. 18, 2021

Related U.S. Application Data
(60) Provisional application No. 62/838,078, filed on Apr. 24, 2019.

(51) Int. Cl. G01N 21/76; G01N 21/64

(52) U.S. Cl. CPC G01N 21/766; G01N 21/64

(57) ABSTRACT

Use of a method of identifying the sources of particulates in the atmosphere, including the steps of; quantifying the amount of polycyclic aromatic hydrocarbons and that of nitropolycyclic aromatic hydrocarbons contained in particulates in the atmosphere; and identifying the ratio of combustion-derived particulates in the atmosphere using logical equations derived in advance, which use the amount of polycyclic aromatic hydrocarbons and that of nitropolycyclic aromatic hydrocarbons contained in pre-quantified particulates derived from different combustion sources, and the amount of polycyclic aromatic hydrocarbons and that of nitropolycyclic aromatic hydrocarbons contained in the particulates in the atmosphere obtained in the step of quantifying, allows identification of the sources of particulates in the atmosphere in which mixed particulates from different sources are suspended.

### TECHNICAL FIELD

The present invention relates to a method for identifying generation sources of fine particulates in the atmosphere. In more detail, the present invention relates to a method for identifying the ratio of combustion sources in the atmosphere by quantifying the amounts of polycyclic aromatic hydrocarbons and nitro compounds thereof contained in particulates derived from different combustion sources in the atmosphere.

### BACKGROUND ART

Air pollution is a growing global concern. The World Health Organization (WHO) reports that ambient air pollution kills millions of people annually.

Among air pollutants, suspended particulate attracts much attention and environmental emission standards for PM₁₀ (particulate with a diameter no more than 10 μm) and PM_{2.5} (particulate with a diameter no more than 2.5 μm) have been set in several countries because of their relationship to mortality and carcinogenicity.

These suspended particulates are largely divided into combustion-derived particulates generated through combustion and other particulates generated by other sources than combustion. The combustion-derived particulates are further divided into combustion-derived particulates at higher temperature and combustion-derived particulates at lower temperature.

It is important to identify the sources of particulates in the atmosphere and develop environment countermeasures corresponding to the sources of the particulates in the atmosphere, so as to observe the environmental emission standards and to implement effective environment countermeasures.

Several methods for identifying generation sources of suspended particulates in the atmosphere have been proposed (e.g., Patent Document 1, Non-Patent Documents 1 to 4).

### PRIOR ART DOCUMENTS

### Patent Documents

Patent Document 1: JP H 07-253420A

### Non-Patent Documents

Non-Patent Document 1: Daisey, J. M., Keyko, M. H., Kneip, T. J., Source identification and allocation of polycyclic aromatic hydrocarbon compounds in the New York city aerosol: methods and applications. In: Jones, P. W., Leber, P. (Eds.), 1979. Polycyclic Aromatic Hydrocarbons, vol. 99. pp. 201-215. Ann Arbor Science. Ann arbor.
Non-Patent Document 2: Masclet, P., Bresson, M. A., Mouvier, G., 1987. Polycyclic aromatic hydrocarbons emitted by power stations and influence of combustion conditions. Fuel, 66, 556-562.
Non-Patent Document 3: Sicre, M. A., Marty, J. C., Saliot, A., Aparicio, X., Grimalt, J., Albaiges, J., 1987. Aliphatic and aromatic hydrocarbons in different sized aerosols over the Mediterranean Sea: occurrence and origin. Atmos. Environ., 21, 2247-2259.
Non-Patent Document 4: Rogge, W. F., Hildemann, L. M., Mazurek, M. A., Cass, G. R., 1993. Simoneit, B. R. T., Sources of fine organic aerosol. 2. Noncatalyst and catalyst-equipped automobiles and heavy-duty diesel trucks. Environ. Sci. Technol., 27, 636-651.

### DISCLOSURE OF THE INVENTION

### Problems to be Solved by the Invention

However, with the above proposed methods, when the atmosphere contains suspended, mixed particulates from different sources, identification of the sources is not accurate, and theoretical proof is weak.

### Means of Solving the Problems

On the other hand, the inventors have focused on polycyclic aromatic hydrocarbons and nitropolycyclic aromatic hydrocarbons contained in particulates in the atmosphere, and have found that the quantitative ratio of polycyclic aromatic hydrocarbons and nitropolycyclic aromatic hydrocarbons contained in combustion-derived particulates at higher temperature is different from that of those contained in combustion-derived particulates at lower temperature, thereby having completed the present invention.

That is, the present invention relates to a method of identifying the sources of particulates in the atmosphere, including the steps of;

quantifying the amount of polycyclic aromatic hydrocarbons and that of nitropolycyclic aromatic hydrocarbons contained in particulates in the atmosphere; and

identifying the ratio of combustion-derived particulates in the atmosphere using logical equations derived in advance, which use the amount of polycyclic aromatic hydrocarbons and that of nitropolycyclic aromatic hydrocarbons contained in pre-quantified particulates derived from different combustion sources, and the amount of polycyclic aromatic hydrocarbons and that of nitropolycyclic aromatic hydrocarbons contained in the particulates in the atmosphere obtained in the step of quantifying.

### Results of the Invention

According to the present invention, even in an atmosphere in which particulates derived from different sources are suspended without knowing the ratio of the particulates derived from different sources in the atmosphere, quantification of the amount of polycyclic aromatic hydrocarbons and that of nitropolycyclic aromatic hydrocarbon compounds contained in the particulates in the atmosphere allows identification of the ratio of particulates derived from the respective sources.

Further according to the present invention, the amounts of polycyclic aromatic hydrocarbons, nitropolycyclic aromatic hydrocarbons, particulates derived from different sources, and particulates derived from sources other than combustion may be found.

### BRIEF DESCRIPTION OF DRAWINGS

FIG. 1 is a graph giving results of Working Example 1; and

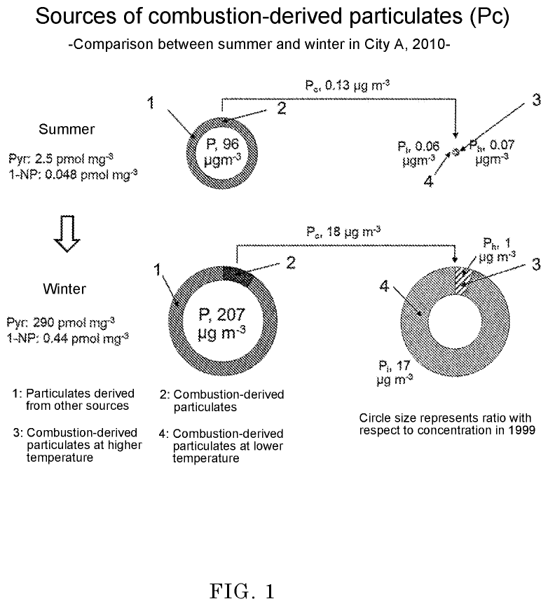

FIG. 2 is a graph giving results of Working Example 2.

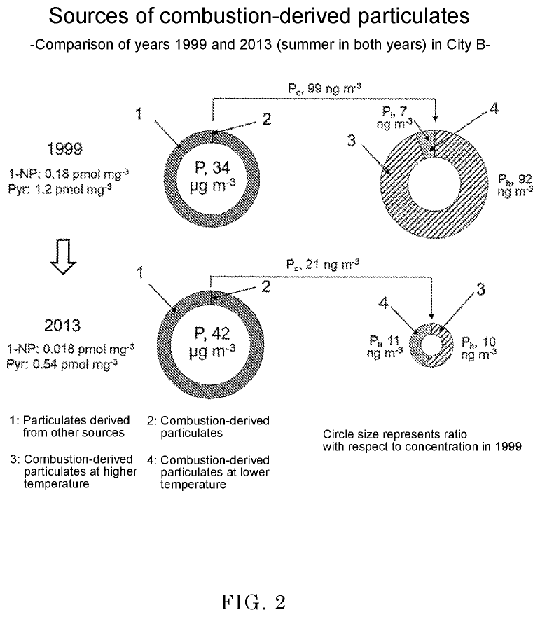

### DESCRIPTION OF EMBODIMENTS

An embodiment according to the present invention is described in detail below.

An identifying method according to the present invention includes the step of quantifying the amounts of polycyclic aromatic hydrocarbons and nitropolycyclic aromatic hydrocarbons contained in particulates in the atmosphere.

The quantifying method is not particularly limited, and a method of separating polycyclic aromatic hydrocarbons and nitropolycyclic aromatic hydrocarbons from the particulates in the atmosphere and measuring the amounts thereof, method of quantifying the amounts of polycyclic aromatic hydrocarbons and nitropolycyclic aromatic hydrocarbons contained in particulates through titration etc., method of separating polycyclic aromatic hydrocarbons and nitropolycyclic aromatic hydrocarbons through chromatography such as liquid chromatography, and measuring the amounts thereof, for example, are available.

Of these quantifying methods, the method of separating polycyclic aromatic hydrocarbons and nitropolycyclic aromatic hydrocarbons contained in particulates through chromatography and measuring the amounts thereof is preferred from the perspective of quantifying capability, and use of liquid chromatography is preferred from the perspective of separability of polycyclic aromatic hydrocarbons and nitropolycyclic aromatic hydrocarbons. High-speed liquid chromatography is preferred from the perspective of measuring speed.

Quantifying the amounts of the separated polycyclic aromatic hydrocarbons and nitropolycyclic aromatic hydrocarbons using fluorescence analysis or chemiluminescence analysis as a detector is especially preferred from the perspective of quantifying capability. It is preferable to separate the polycyclic aromatic hydrocarbons and the nitropolycyclic aromatic hydrocarbons extracted from the particulates in the atmosphere using high-speed liquid chromatography, for example, and to quantify the amounts of the separated chemical compounds through fluorescence analysis or chemiluminescence analysis. Analysis and quantification of the polycyclic aromatic hydrocarbons may be carried out through gas chromatography and mass spectroscopy (GC-MS). Use of nothing but an analysis apparatus capable of carrying out fluorescence analysis and chemiluminescence analysis is preferred from the perspective that measurement of polycyclic aromatic hydrocarbons and nitropolycyclic aromatic hydrocarbons can be carried out simultaneously.

In the case of quantification through chromatography, use of an internal standard reagent is preferred. As the internal standard reagent used in fluorescence analysis and chemiluminescence analysis, a deuterated polycyclic aromatic hydrocarbon such as pyrene-d₁₀ or benzo[a]pyrene-d₁₂ is used for the polycyclic aromatic hydrocarbon, and 2-fluoro-7-nitrofluoren or 1-nitropyrene-d₉ is used for the nitropolycyclic aromatic hydrocarbon, for example.

Note that the amounts of particulates in the atmosphere to be quantified is not particularly limited, and that the amounts of particulates required for quantification may be collected from the atmosphere. In the case of a combination of the high-speed liquid chromatography described above and fluorescence analysis and chemiluminescence analysis, 0.01 mg to 100 mg, preferably 0.05 mg to 3 mg of particulates should be collected from the atmosphere.

The method of collecting the particulates from the atmosphere is not particularly limited, and air should be suctioned and collected into a cylinder etc., and particulates should be separated from the collected air using a filter and/or centrifugal separation, etc., and then be subjected to quantitative analysis. A quartz fiber filter and a glass fiber filter are examples of the filter. Polycyclic aromatic hydrocarbons and nitropolycyclic aromatic hydrocarbons are extracted from the separated particulates using an organic solvent etc., and are then cleaned up using a liquid-liquid method, a column-cartridge method, etc. A part thereof is injected into a high-speed liquid chromatography apparatus, so as to conduct quantitative analysis.

Use of the amounts of polycyclic aromatic hydrocarbons and nitropolycyclic aromatic hydrocarbons contained in the particulates in the atmosphere obtained through the method described above, and the amounts of polycyclic aromatic hydrocarbons and nitropolycyclic aromatic hydrocarbons contained in pre-quantified particulates derived from different combustion sources allows identification of sources of the particulates in the atmosphere.

Typically, the particulates in the atmosphere are divided into combustion-derived particulates derived from combustion facilities and other particulates derived from other sources. Moreover, combustion facilities may be divided into high-temperature combustion facilities and low-temperature combustion facilities, and particulates generated by the respective facilities may be divided into high-temperature combustion-derived particulate s and low-temperature combustion-derived particulate s. High-temperature combustion facilities are generally combustion facilities using combustion temperatures of 2000° C. or higher, such as a diesel engine or a gasoline engine. Combustion temperatures in the diesel engine and the gasoline engine are 2700 to 3000° C.

On the other hand, low-temperature combustion facilities are generally combustion facilities using combustion temperatures less than 2000° C., such as a coal stove or a coal boiler. Combustion temperatures in the coal stove and the coal boiler are 1100 to 1200° C. In addition to wood-burning stoves, biomass-burning such as rice straw burning, slash and burn agriculture, forest fires, etc. are also low-temperature combustion facilities using combustion temperatures of 500 to 600° C.

By quantifying in advance the amounts of the polycyclic aromatic hydrocarbons and the nitropolycyclic aromatic hydrocarbons contained in the particulates collected from the combustion facilities described above, the compositional ratio of the polycyclic aromatic hydrocarbons and the nitropolycyclic aromatic hydrocarbons in the combustion-derived particulates at higher temperature, and that of the same in the combustion-derived particulates at lower temperature may be found.

The amounts of polycyclic aromatic hydrocarbons and nitropolycyclic aromatic hydrocarbons contained in the particulates derived from different combustion sources may be found using numerical values already presented in articles etc., or by quantifying separately the amounts of polycyclic aromatic hydrocarbons and nitropolycyclic aromatic hydrocarbons contained in particulates derived from known combustion sources and using those quantified values.

Since the particulates in the atmosphere are mixed with particulates generated from different combustion sources, the ratio of particulates derived from the respective sources cannot be measured directly. However, if the ratio of polycyclic aromatic hydrocarbons to nitropolycyclic aromatic hydrocarbons contained in the particulates derived from different sources is known, the ratio of sources of the particulates in the atmosphere may be reached using logical equations derived in advance.

Of these logical equations, a logical equation including the amount of polycyclic aromatic hydrocarbons in the atmosphere, amount of nitropolycyclic aromatic hydrocarbons in the atmosphere, and quantitative ratio thereof is convenient for identifying the sources of the particulates.

Logical equations including the following equations (1) and (2) are given as exemplary logical equations.

[NP]/[P]={[NPh]x+[NPl](1-x)}/{[Ph]x+[Pl](1-x)} (1)

[NP]={[NPh]x+[NPl](1-x)}y (2)

where [P] and [NP] represent respective concentrations of polycyclic aromatic hydrocarbons and nitropolycyclic aromatic hydrocarbons contained in the particulates in the atmosphere, [Ph] and [NPh] represent respective concentrations of polycyclic aromatic hydrocarbons and nitropolycyclic aromatic hydrocarbons contained in pre-quantified particulates derived from high-temperature combustion sources, and [Pl] and [NPl] represent respective concentrations of polycyclic aromatic hydrocarbons and nitropolycyclic aromatic hydrocarbons contained in pre-quantified particulates derived from low-temperature combustion sources. These values are either literature data, as described above, or values obtained through separate measurement and quantification.

‘x’ denotes the ratio of combustion-derived particulates at higher temperature included in the combustion-derived particulates (where 0<x<1), and ‘y’ denotes the ratio of combustion-derived particulates included in the particulates in the atmosphere (where 0<y<1). Therefore, the ratio of the combustion-derived particulates at lower temperature included in the combustion-derived particulates is (1-x), and ratio of the other particulates included in the particulates in the atmosphere is (1-y).

Finding x and y from the equations (1) and (2) given above allows identification of the ratio of sources of the particulates in the atmosphere.

Moreover, since polycyclic aromatic hydrocarbons and nitropolycyclic aromatic hydrocarbons are considered to be main constituents of the combustion-derived particulates, the following logical equations are derived when the ratio of nitropolycyclic aromatic hydrocarbons generated due to high-temperature combustion to nitropolycyclic aromatic hydrocarbons in the atmosphere is a (where 0<a<1), and ratio of polycyclic aromatic hydrocarbons generated due to high-temperature combustion to polycyclic aromatic hydrocarbons in the atmosphere is b (where 0<b<1). Note that the ratio of nitropolycyclic aromatic hydrocarbons generated due to low-temperature combustion is (1-a), and ratio of polycyclic aromatic hydrocarbons generated due to high-temperature combustion is (1-b).

[NPh]/[Ph]=[NP]a/[P]b (3)

[NPl]/[Pl]={[NP](1-a)/[Pl](1-b) (4)

a and b are found by solving equations (3) and (4) given above using either literature data or values obtained through separate measurement and quantification as the values of [Ph], [NPh], [Pl] and [NPl], as described above, resulting in identification of the ratio of the sources of polycyclic aromatic hydrocarbons and nitropolycyclic aromatic hydrocarbons in the atmosphere.

While types of polycyclic aromatic hydrocarbons contained in the particulates in the atmosphere to be quantified are not particularly limited, it is preferable that they are present in the atmosphere at a relatively high concentration, and that the 16 types of polycyclic aromatic hydrocarbons specified by the U.S. Environmental Protection Agency from the perspective of effect on the human body are quantified. The 16 types of compounds are given below. At least one type of these polycyclic aromatic hydrocarbons should be quantified.

The 16 types of polycyclic aromatic hydrocarbons are naphthalene, acenaphthene, fluorene, phenanthrene, anthracene, fluoranthene, pyrene, benz[a]anthracene, chrysene, benzo[b]fluoranthene, benzo[k]fluoranthene, benzo[a]pyrene, dibenz[a,h]anthracene, benzo[ghi]perylene, and indeno[1,2,3-cd]pyrene. Structural formulas of these compounds are as given below.

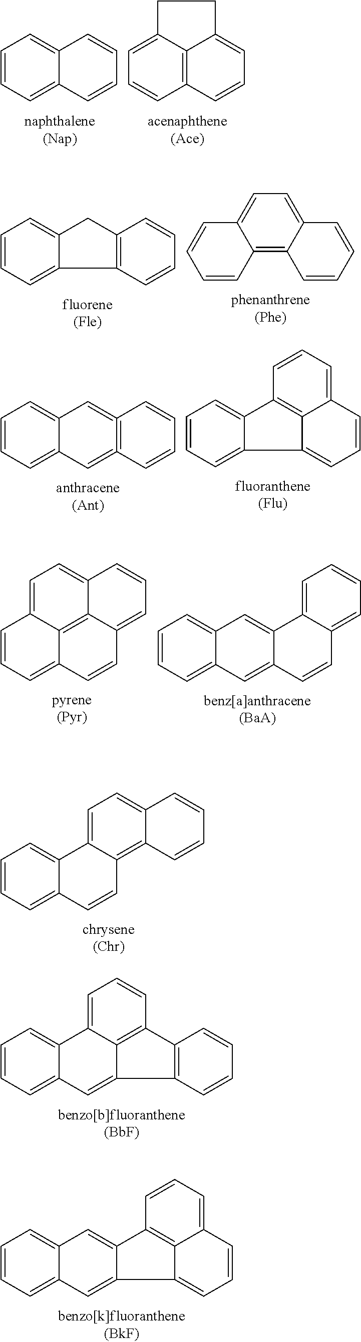

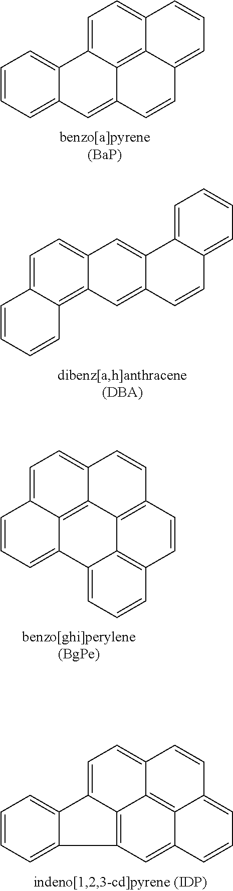

While types of polycyclic aromatic hydrocarbons contained in the particulates derived from different combustion sources to be quantified in advance are also not particularly limited, the same polycyclic aromatic hydrocarbons as given above may be used, for example. When the combustion sources are different, the same type of polycyclic aromatic hydrocarbons may be quantified, or otherwise different polycyclic aromatic hydrocarbons may be quantified. From the perspective of convenience when identifying the sources using the logical equations, quantifying the same polycyclic aromatic hydrocarbons is preferred. The polycyclic aromatic hydrocarbons to be quantified may be one type or two or more types.

Moreover, the polycyclic aromatic hydrocarbons contained in the particulates in the atmosphere to be quantified and polycyclic aromatic hydrocarbons contained in the pre-quantified particulates derived from different combustion sources may be the same or different compounds; however, from the perspective of convenience when identifying sources using the logical equations, quantifying the same polycyclic aromatic hydrocarbons is preferred.

While types of nitropolycyclic aromatic hydrocarbons contained in the particulates in the atmosphere to be quantified are not particularly limited, quantifying the amounts of the 14 types of polycyclic aromatic hydrocarbons specified by the U.S. Environmental Protection Agency from the perspective of effect on the human body is preferred. The 13 types of compounds are given below. At least one type of these polycyclic aromatic hydrocarbons should be quantified.

The 13 types of nitropolycyclic aromatic hydrocarbons are dinitropyrene, nitropyrene, nitrobenzanthrone, 2-nitrofluorene, nitroanthracene, 5-nitroacenaphthene, nitrophenanthrene, 3-nitrofluoranthene, 7-nitrobenz[a]anthracene, 2-nitrotriphenylene, 6-nitrochrysene, 6-nitrobenzo[a]pyrene, and nitroperylene. Structural formulas of these compounds are as given below. Note that in the case where there is an isomer with a distinct nitro group substitution arrangement, only representative compounds are given.

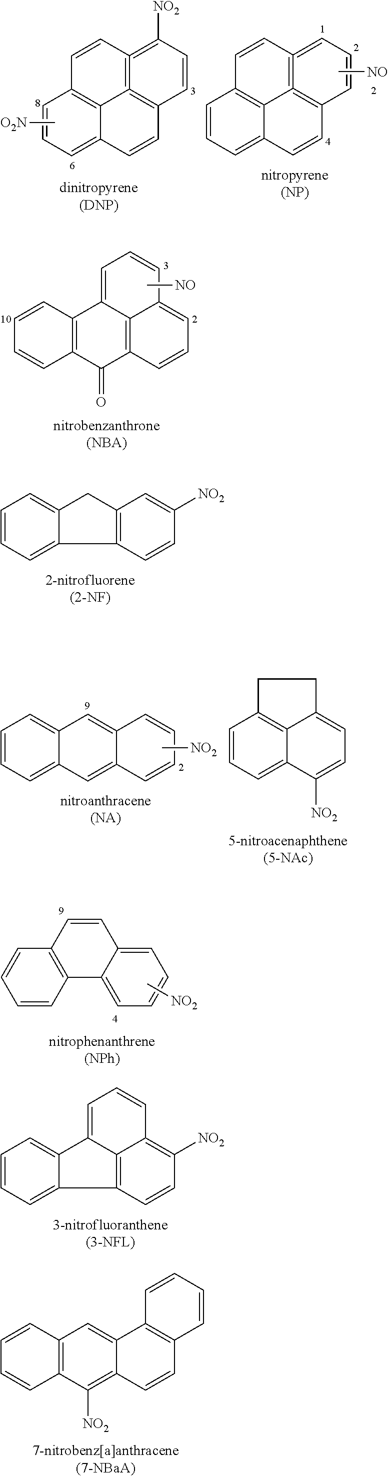

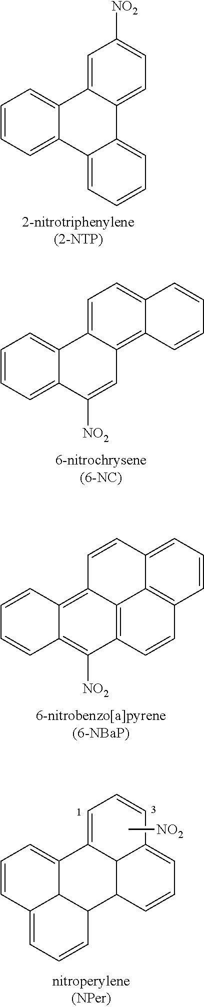

While types of nitropolycyclic aromatic hydrocarbons contained in the pre-quantified particulates derived from different combustion sources are also not particularly limited, the same nitropolycyclic aromatic hydrocarbons as given above may be used, for example. When the combustion sources are different, the same type of nitropolycyclic aromatic hydrocarbons may be quantified, or otherwise different nitropolycyclic aromatic hydrocarbons may be quantified. From the perspective of convenience when identifying sources using the logical equations, quantifying the same nitropolycyclic aromatic hydrocarbons is preferred. The nitropolycyclic aromatic hydrocarbons to be quantified may be one type or two or more types.

Moreover, the nitropolycyclic aromatic hydrocarbons contained in the particulates in the atmosphere to be quantified and nitropolycyclic aromatic hydrocarbons contained in the pre-quantified particulates derived from different combustion sources may be the same or different compounds; however, from the perspective of convenience when identifying sources using the logical equations, quantifying the same polycyclic aromatic hydrocarbons is preferred.

Furthermore, in the case of quantifying polycyclic aromatic hydrocarbons and nitropolycyclic aromatic hydrocarbons, it is preferable, from the perspective of convenience when identifying sources using the logical equations, that the nitropolycyclic aromatic hydrocarbons are compounds formed through nitration of the polycyclic aromatic hydrocarbons to be quantified.

For example, the compounds described above may be combinations of acenaphthene and nitroacenaphthene, fluorene and nitrofluorene, phenanthrene and nitrophenanthrene, anthracene and nitroanthracene, fluoranthene and nitrofluoranthen, pyrene and nitropyrene, benz[a]anthracene and nitrobenz[a]anthracene, chrysene and nitrochrysene, etc.

Of these combinations, the combination of pyrene and nitropyrene is preferred from the perspective of quantifying capability.

Sources of particulates in the atmosphere may be identified using the method described above.

### WORKING EXAMPLES

Results of identifying the sources of particulates in the atmosphere in cities using the logical equations (1), (2), (3), and (4) given above are described below.

In the following working examples, pyrene (Pyr) is used as the polycyclic aromatic hydrocarbons and nitropyrene (1-NP) is used as the nitropolycyclic aromatic hydrocarbons. Moreover, the values taken from a document (Tang, N. et. al., Atoms. Environ. (2005)) already reported are respectively substituted for [Ph], [NPh], [Pl] and [NPl] in the logical equations (1), (2), (3), and (4). The respective values are as given below, where unit is pmol mg⁻³.
[Ph]=180
[NPh]=65.5
[Pl]=3500
[NPl]=1.43

Quantification of polycyclic aromatic hydrocarbons and nitropolycyclic aromatic hydrocarbons contained in the particulates is carried out through the steps described above.

### Working Example 1

Amounts of pyrene and nitropyrene contained in particulates in the atmosphere of City A in the winter and summer of 2010 are quantified. The amounts of pyrene are 2.5 pmol mg⁻³ in the summer and 290 pmol mg⁻³ in the winter, and amounts of nitropyrene are 0.048 pmol mg⁻³ in the summer and 0.44 pmol mg⁻³ in the winter. The amounts of particulates in the atmosphere are 96 pmol mg⁻³ in the summer and 207 pmol mg⁻³ in the winter. Resulting values of x, y, a and b using the above logical equations are given in Table 1.

### Working Example 2

Amounts of pyrene and nitropyrene contained in particulates in the atmosphere of City B in the summer of 1999 and the summer of 2013 are quantified. The amounts of pyrene are 1.2 pmol mg⁻³ in 1999 and 0.54 pmol mg⁻³ in 2013, and amounts of nitropyrene are 0.18 pmol mg⁻³ in 1999 and 0.018 pmol mg⁻³ in 2013. The amounts of particulates in the atmosphere are 34 pmol mg⁻³ in 1999 and 42 pmol mg⁻³ in 2013. Resulting values of x, y, a and b using the above logical equations are given in Table 1.

TABLE 1

| | | | |
|---|---|---|---|
| a | b | x | y |

Working Example 1

| | | | | | |
|---|---|---|---|---|---|
| 2010 | Summer | 0.979 | 0.052 | 0.515 | 0.0014 |
| | Winter | 0.731 | 0.003 | 0.056 | 0.088 |

Working Example 2

| | | | | | |
|---|---|---|---|---|---|
| 1999 | Summer | 0.997 | 0.149 | 0.932 | 0.0029 |
| 2013 | Summer | 0.993 | 0.0032 | 0.539 | 0.00050 |

From the results given above, the combustion-derived particulates have increased in the winter of City A, which is considered mostly a consequence of increase in coal-heating exhaust (of combustion-derived particulates at lower temperature). On the other hand, when comparing City B in 1999 and 2013, the combustion-derived particulates have decreased, which is understood as mostly a consequence of decrease in automobile emission (of combustion-derived particulates at higher temperature).

[Aspects of Invention]

The working examples described above are understood by those skilled in the art to be specific examples of the following aspects.

[Item 1] A method of identifying the sources of particulates in the atmosphere, including the steps of;

quantifying the amount of polycyclic aromatic hydrocarbons and that of nitropolycyclic aromatic hydrocarbons contained in particulates in the atmosphere; and

identifying the ratio of combustion-derived particulates in the atmosphere using logical equations derived in advance, which use the amount of polycyclic aromatic hydrocarbons and that of nitropolycyclic aromatic hydrocarbons contained in pre-quantified particulates derived from different combustion sources and the amount of polycyclic aromatic hydrocarbons and that of nitropolycyclic aromatic hydrocarbons contained in the particulates in the atmosphere obtained in the step of quantifying.

According to the method of identifying of Item 1, even if the atmosphere has suspended particulates derived from different sources, and the ratio of the particulates derived from different sources in the atmosphere is unknown, the ratio of the particulates derived from the respective sources may be identified by quantifying the amount of polycyclic aromatic hydrocarbons and that of nitropolycyclic aromatic hydrocarbons contained in the particulates in the atmosphere.

[Item 2] The method of identifying according to Item 1, further including the step of quantifying the amount of polycyclic aromatic hydrocarbons and that of nitropolycyclic aromatic hydrocarbons contained in the particulates derived from multiple different combustion sources.

[Item 3] The method of identifying according to either Item 1 or Item 2, wherein one of the different combustion sources is a high-temperature combustion facility.

[Item 4] The method of identifying according to any one of Item 1 to Item 3, wherein one of the different combustion sources is a low-temperature combustion facility.

[Item 5] The method of identifying according to any one of Items 1 to 4, wherein the logical equations derived in advance are logical equations including the concentration of the polycyclic aromatic hydrocarbons in the atmosphere, concentration of the nitropolycyclic aromatic hydrocarbons in the atmosphere, and quantitative ratio of the concentrations.

[Item 6] The method of identifying according to any one of Item 1 to Item 5, wherein the logical equations derived in advance are the following equations (1) and (2):

[NP]/[P]={[NPh]x+[NPl](1-x)}/{[Ph]x+[Pl](1-x)} (1)

[NP]={[NPh]x+[NPl](1-x)}y (2)

where [P] and [NP] represent respective concentrations of polycyclic aromatic hydrocarbons and nitropolycyclic aromatic hydrocarbons contained in the particulates in the atmosphere, [Ph] and [NPh] represent respective concentrations of polycyclic aromatic hydrocarbons and nitropolycyclic aromatic hydrocarbons contained in pre-quantified particulates derived from high-temperature combustion sources, and [Pl] and [NPl] represent respective concentrations of polycyclic aromatic hydrocarbons and nitropolycyclic aromatic hydrocarbons contained in pre-quantified particulates derived from low-temperature combustion sources. ‘x’ denotes the ratio of combustion-derived particulates at higher temperature included in the combustion-derived particulates (where 1<x<0), and ‘y’ denotes the ratio of combustion-derived particulates included in the particulates in the atmosphere (where 1<y<0).

[Item 7] The method of identifying according to any one of Items 1, 5, and 6, wherein the logical equations derived in advance are the following equations (3) and (4):

[NPh]/[Ph]=[NP]a/[P]b (3)

[NPl]/[Pl]={[NP](1-a)/[Pl](1-b) (4)

where [P], [NP], [Ph], [NPh], [Pl], and [NPl] are the same as those claimed in Item 6, ‘a’ denotes the ratio of nitropolycyclic aromatic hydrocarbons derived from high-temperature combustion sources to nitropolycyclic aromatic hydrocarbons in the atmosphere (where 0<a<1), and ‘b’ denotes the ratio of polycyclic aromatic hydrocarbons derived from high-temperature combustion sources to polycyclic aromatic hydrocarbons in the atmosphere.

[Item 8] The method of identifying according to Item 3, wherein the high-temperature combustion facility is a gasoline engine or a diesel engine.

[Item 9] The method of identifying according to Item 4, wherein the low-temperature combustion facility is a coal boiler or a coal stove.

L00571 [Item 10] The method of identifying according to any one of Item 1 to Item 9, wherein the polycyclic aromatic hydrocarbons in the atmosphere and the polycyclic aromatic hydrocarbons contained in the multiple different combustion sources are the same.

[Item 11] The method of identifying according to any one of Item 1 to Item 10, wherein the nitropolycyclic aromatic hydrocarbons in the atmosphere and the nitropolycyclic aromatic hydrocarbons contained in the multiple different combustion sources are the same.

[Item 12] The method of identifying according to any one of Item 1 to Item 11, wherein the nitropolycyclic aromatic hydrocarbons in the atmosphere are formed through nitration of the polycyclic aromatic hydrocarbons in the atmosphere.

[Item 13] The method of identifying according to any one of Item 1 to Item 12, wherein the nitropolycyclic aromatic hydrocarbons contained in the multiple different combustion sources are formed through nitration of the polycyclic aromatic hydrocarbons contained in the multiple different combustion sources.

[Item 14] The method of identifying according to any one of Items 1, 10, and 13, wherein the polycyclic aromatic hydrocarbons are at least one selected from a group consisting of naphthalene, acenaphthene, fluorene, phenanthrene, anthracene, fluoranthene, pyrene, benz[a]anthracene, chrysene, benzo[b]fluoranthene, benzo[k]fluoranthene, benzo[a]pyrene, dibenz[a,h]anthracene, benzo[ghi]perylene, and indeno[1,2, 3-cd]pyrene.

[Item 15] The method of identifying according to any one of Items 1, 11, 12 and 13, wherein the nitropolycyclic aromatic hydrocarbons are at least one selected from a group consisting of dinitropyrene, nitropyrene, nitrobenzanthrone, 2-fluoro-7-nitrofluoren, 2-nitrofluorene, nitroanthracene, 5-nitroacenaphthene, nitrophenanthrene, 3-nitrofluoranthene, 7-nitrobenz[a]anthracene, 2-nitrotriphenylene, 6-nitrochrysene, 6-nitrobenzo[a]pyrene, and nitroperylene.

[Item 16] The method of identifying according to either Item 14 or Item 15, wherein the polycyclic aromatic hydrocarbons are perylene, and the nitropolycyclic aromatic hydrocarbons are nitroperylene.

[Item 17] The method of identifying of Item 1, wherein quantifying the amount of polycyclic aromatic hydrocarbons and that of nitropolycyclic aromatic hydrocarbons contained in the particulates in the atmosphere is carried out using fluorescence analysis and chemiluminescence analysis.

[Item 18] The method of identifying according to Item 17, wherein the fluorescence analysis and the chemiluminescence analysis are carried out simultaneously.

### DESCRIPTION OF REFERENCES

1: Ratio of particulates derived from other sources
2: Ratio of combustion-derived particulates
3: Ratio of combustion-derived particulates at higher temperature
4: Ratio of combustion-derived particulates at lower temperature

1. A method of identifying generation sources of particulates in the atmosphere, comprising the steps of:
quantifying the amount of polycyclic aromatic hydrocarbons and that of nitropolycyclic aromatic hydrocarbons contained in particulates in the atmosphere; and
identifying the ratio of combustion-derived particulates in the atmosphere using logical equations derived in advance, which use the amount of polycyclic aromatic hydrocarbons and that of nitropolycyclic aromatic hydrocarbons contained in pre-quantified particulates derived from different combustion sources and the amount of polycyclic aromatic hydrocarbons and that of nitropolycyclic aromatic hydrocarbons contained in the particulates in the atmosphere obtained in the step of quantifying.

2. The method of identifying according to claim 1, further comprising the step of quantifying the amount of polycyclic aromatic hydrocarbons and that of nitropolycyclic aromatic hydrocarbons contained in particulates derived from multiple different combustion sources.

3. The method of identifying according to claim 1, wherein one of the different combustion sources is a high-temperature combustion facility.

4. The method of identifying according to claim 1, wherein one of the different combustion sources is a low-temperature combustion facility.

5. The method of identifying according to claim 1, wherein the logical equations derived in advance are logical equations including the concentration of the polycyclic aromatic hydrocarbons in the atmosphere, concentration of the nitropolycyclic aromatic hydrocarbons in the atmosphere, and quantitative ratio of the concentrations.

6. The method of identifying according to claim 1, wherein the logical equations derived in advance are the following equations (1) and (2):
[NP]/[P]={[NPh]x+[NPl](1-x)}/{[Ph]x+[Pl](1-x)} (1)
[NP]={[NPh]x+[NPl](1-x)}y (2)
where [P] and [NP] represent respective concentrations of polycyclic aromatic hydrocarbons and nitropolycyclic aromatic hydrocarbons contained in the particulates in the atmosphere, [Ph] and [NPh] represent respective concentrations of polycyclic aromatic hydrocarbons and nitropolycyclic aromatic hydrocarbons contained in pre-quantified particulates derived from high-temperature combustion sources, and [Pl] and [NPl] represent respective concentrations of polycyclic aromatic hydrocarbons and nitropolycyclic aromatic hydrocarbons contained in pre-quantified particulates derived from low-temperature combustion sources; and ‘x’ denotes the ratio of combustion-derived particulates at higher temperature included in the combustion-derived particulates (where 0<x<1), and ‘y’ denotes the ratio of combustion-derived particulates included in the particulates in the atmosphere (where 0<y<1).

7. The method of identifying according to claim 1, wherein the logical equations derived in advance include the following equations (3) and (4):
[NPh]/[Ph]={[NP]a}/{[P]b} (3)
[NPl]/[Pl]={[NP](1-a)}/{[Pl](1-b)} (4)
where [P] and [NP] represent respective concentrations of polycyclic aromatic hydrocarbons and nitropolycyclic aromatic hydrocarbons contained in the particulates in the atmosphere, [Ph] and [NPh] represent respective concentrations of polycyclic aromatic hydrocarbons and nitropolycyclic aromatic hydrocarbons contained in pre-quantified particulates derived from high-temperature combustion sources, and [Pl] and [NPl] represent respective concentrations of polycyclic aromatic hydrocarbons and nitropolycyclic aromatic hydrocarbons contained in pre-quantified particulates derived from low-temperature combustion sources; and ‘a’ denotes the ratio of nitropolycyclic aromatic hydrocarbons derived from high-temperature combustion sources to nitropolycyclic aromatic hydrocarbons in the atmosphere (where 0<a<1), and ‘b’ denotes ratio of polycyclic aromatic hydrocarbons derived from high-temperature combustion sources to polycyclic aromatic hydrocarbons in the atmosphere (where 0<b<1).

8. The method of identifying according to claim 3, wherein the high-temperature combustion facility is a gasoline engine or a diesel engine.

9. The method of identifying according to claim 4, wherein the low-temperature combustion facility is a coal boiler or a coal stove.

10. The method of identifying according to claim 1, wherein the polycyclic aromatic hydrocarbons in the atmosphere and the polycyclic aromatic hydrocarbons contained in the multiple different combustion sources are the same.

11. The method of identifying according to claim 1, wherein the nitropolycyclic aromatic hydrocarbons in the atmosphere and the nitropolycyclic aromatic hydrocarbons contained in the multiple different combustion sources are the same.

12. The method of identifying according to claim 1, wherein the nitropolycyclic aromatic hydrocarbons in the atmosphere are formed through nitration of the polycyclic aromatic hydrocarbons in the atmosphere.

13. The method of identifying according to claim 1, wherein the nitropolycyclic aromatic hydrocarbons contained in the multiple different combustion sources are formed through nitration of the polycyclic aromatic hydrocarbons contained in the multiple different combustion sources.

14. The method of identifying according to claim 1, wherein the polycyclic aromatic hydrocarbons are at least one selected from a group consisting of naphthalene, acenaphthene, acenaphthylene, fluorene, phenanthrene, anthracene, fluoranthene, pyrene, benz[a]anthracene, chrysene, benzo[b]fluoranthene, benzo[k]fluoranthene, benzo[a]pyrene, dibenz[a,h]anthracene, benzo[ghi]perylene, and indeno[1,2,3-cd]pyrene.

15. The method of identifying according to claim 1, wherein the nitropolycyclic aromatic hydrocarbons are at least one selected from a group consisting of dinitropyrene, nitropyrene, nitrobenzanthrone, 2-fluoro-7-nitrofluoren, 2-nitrofluorene, nitroanthracene, 5-nitroacenaphthene, nitrophenanthrene, 3-nitrofluoranthene, 7-nitrobenz[a]anthracene, 2-nitrotriphenylene, 6-nitrochrysene, 6-nitrobenzo[a]pyrene, and nitroperylene.

16. The method of identifying according to claim 14, wherein the polycyclic aromatic hydrocarbons are perylene, and the nitropolycyclic aromatic hydrocarbons are nitroperylene.

17. The method of identifying according to claim 1, wherein quantifying the amount of polycyclic aromatic hydrocarbons and that of nitropolycyclic aromatic hydrocarbons contained in the particulates in the atmosphere is carried out through fluorescence analysis and chemiluminescence analysis.

18. The method of identifying according to claim 17, wherein the fluorescence analysis and the chemiluminescence analysis are carried out simultaneously.
